(19) United States
(12) Patent Application Publication
(10) Pub. No.: US 2024/0008859 A1
(43) Pub. Date: Jan. 11, 2024

(54) POROUS BODY FOR CAPTURING CANCER CELLS

(71) Applicants: NIHON KOHDEN CORPORATION, Tokyo (JP); NATIONAL UNIVERSITY CORPORATION TOKAI NATIONAL HIGHER EDUCATION AND RESEARCH SYSTEM, Nagoya-shi, Aichi (JP); UNIVERSITY PUBLIC CORPORATION OSAKA, Osaka-shi, Osaka (JP)

(72) Inventors: Shinya NAGATA, Tokorozawa-shi, Saitama (JP); Akane SUZUKI, Tokorozawa-shi, Saitama (JP); Takahiro SHIOYAMA, Tokorozawa-shi, Saitama (JP); Tadanori SUGIMOTO, Tokorozawa-shi, Saitama (JP); Yukino SHINOZAKI, Tokorozawa-shi, Saitama (JP); Masakazu SATSU, Tokorozawa-shi, Saitama (JP); Chikara OHTSUKI, Nagoya-shi, Aichi (JP); Jin NAKAMURA, Nagoya-shi, Aichi (JP); Hideo AKIYOSHI, Sakai-shi, Osaka (JP); Hidetaka NISHIDA, Sakai-shi, Osaka (JP); Keiichiro MIE, Sakai-shi, Osaka (JP)

(73) Assignees: NIHON KOHDEN CORPORATION, Tokyo (JP); NATIONAL UNIVERSITY CORPORATION TOKAI NATIONAL HIGHER EDUCATION AND RESEARCH SYSTEM, Nagoya-shi, Aichi (JP); UNIVERSITY PUBLIC CORPORATION OSAKA, Osaka-shi, Osaka (JP)

(21) Appl. No.: 18/039,171

(22) PCT Filed: Nov. 29, 2021
(86) PCT No.: PCT/JP2021/043702

§ 371 Date: May 26, 2023

(30) Foreign Application Priority Data
Nov. 30, 2020 (JP) 2020-198470

(51) Int. Cl. A61B 10/02; A61F 2/01; A61L 31/02; A61L 31/12; A61L 31/14

(52) U.S. Cl. CPC A61B 10/02; A61F 2/01; A61L 31/026; A61L 31/124; A61L 31/146

(57) ABSTRACT

Provided is a porous body for capturing cancer cells, including a biocompatible inorganic material, the porous body for capturing cancer cells having biocompatibility and also having stability in a living body. The porous body for capturing cancer cells can be used for application related to cancer such as a treatment, a treatment assistance, a test, or a diagnosis.

### TECHNICAL FIELD

The present invention relates to a porous body for capturing cancer cells.

### BACKGROUND ART

The leading cause of death for Japanese people has been a malignant tumor, so-called “cancer” since 1981, and currently, not less than 300,000 people die of cancer a year, and not less than 600,000 people newly develop cancer a year. In future in Japan, it is predicted that as the ageing of the population advances, the number of cancer patients is increasing more and more.

An early detection of cancer cells is one of the goals for optimal treatment and diagnosis. However, detection of cancer cells before metastasis or spread of the cancer cells occurs is sometimes difficult, and even after metastasis or spread of the cancer cells occurs, cancer cells are sometimes not detected. Thus, if an operation cannot be performed or cancer cannot be cured even if an operation is performed, due to the reason that the detection of cancer cells and the diagnosis are delayed and cancer cells systemically metastasize, a treatment such as chemotherapy with an anticancer agent or special radiotherapy is performed.

For the mechanism of cancer metastasis, the processes of a release of cancer cells from a primary lesion, drift to a target organ via blood flow and lymph flow and by migration in a tissue, invasion in a tissue and proliferation of a cancer tissue are known to follow. In the treatment of cancer, inhibition of any of these processes has been tried. For example, Patent Document 1 discloses a technique for suppressing metastasis of cancer to a target organ by capturing circulating tumor cells (CTCs) using a porous body containing polycaprolactone.

### CITATION LIST

### Patent Literature

[PTL 1] U.S. Patent Application Publication No. 2019/0008971

### SUMMARY OF INVENTION

### Technical Problem

However, the porous body described in Patent Document 1 is produced from polycaprolactone that is a biodegradable material. Thus, when it is implanted over a long period of time, the porous body having captured metastatic cancer cells is biodegraded, and the captured cancer cells may be released in a living body.

The present invention has been made in view of such circumstances, and an object thereof is to provide a porous body for capturing cancer cells having biocompatibility and also having stability in a living body.

### Solution to Problem

The present inventors have conducted intensive studies in view of the above problems, and as a result, they found that the above problems are solved by a porous body for capturing cancer cells containing a biocompatible inorganic material, and thus completed the present invention.

### Advantageous Effects of Invention

The present invention provides a porous body for capturing cancer cells having biocompatibility and also having stability in a living body.

### BRIEF DESCRIPTION OF DRAWINGS

FIG. 1 is an optical micrograph illustrating the result of the cell adhesion test in Test Example 1 in Examples.

FIG. 2 is a scanning electron micrograph illustrating the result of the cell adhesion test in Test Example 1 in Examples.

FIG. 3 shows optical micrographs illustrating the results of a cell adhesion test in Test Example 2 in Examples (left view: a porous body for capturing cancer cells, right view: a smooth and dense scaffold).

FIG. 4 shows scanning electron micrographs illustrating the results of the cell adhesion test in Test Example 2 in Examples (left view: a porous body for capturing cancer cells, right view: a smooth and dense scaffold).

FIG. 5A shows an optical micrograph of a porous body for capturing cancer cells (upper view) and the result of the surface roughness of the porous body for capturing cancer cells (lower view).

FIG. 5B shows an optical micrograph of a smooth and dense scaffold (upper view), and the result of the surface roughness of the smooth and dense scaffold (lower view).

FIG. 6 shows a schematic view (upper view) of a procedure in Test Example 3 in Examples, and a photograph (lower view) of a mouse into which the porous body for capturing cancer cells is implanted in Test Example 3 in Examples.

FIG. 7 is a graph illustrating the results of the evaluation of the life extension effect on cancer-bearing mice into which the porous body for capturing cancer cells is implanted in Test Example 4 in Examples.

### DESCRIPTION OF EMBODIMENTS

Hereinafter, embodiments according to one aspect of the present invention will be described. The present invention is not limited only to the following embodiments.

In the present description, the expression “X to Y”, which indicates a range, means “X or more and Y or less”. Further, unless otherwise specified, the operation and the measurement of a physical property or the like are performed under the conditions of room temperature (20 to 25° C.) and a relative humidity of 40 to 50% RH.

<Porous Body for Capturing Cancer Cells>

One embodiment of the present invention relates to a porous body for capturing cancer cells containing a biocompatible inorganic material. In the present description, “porous body for capturing cancer cells” is also simply referred to as “porous body of this embodiment”.

In the present description, “capturing cancer cells” means that cancer cells adhere to a side face or the inside of a pore of the porous body.

In the present description, “porous body” means a solid substance or a structure including many pores.

In the present description, “biocompatibility” means the attribution of a material capable of coexisting with a living body while fulfilling its original function without adversely affecting the living body and without giving a strong stimulus to the living body over a long period of time.

The biocompatible inorganic material according to this embodiment is not limited as long as it is an inorganic material having the above-mentioned biocompatibility. Further, the biocompatible inorganic material may be crystalline or amorphous, and may be inorganic glass as an example of an amorphous material. The use of the biocompatible inorganic material allows for suppressing an adverse event and degradation in a living body to achieve its stability.

Examples of the biocompatible inorganic material include yttria-stabilized zirconia, silica, a silicate compound, a phosphate compound (for example, a calcium phosphate compound), alumina, mullite, and a metallic material (for example, titanium, a titanium alloy, a shape memory alloy such as Ni—Ti, an aluminum alloy, a cobalt-chromium alloy and stainless steel).

In one embodiment, the biocompatible inorganic material is preferably yttria-stabilized zirconia. The yttria-stabilized zirconia preferably contains yttria in an amount of 1 to 20 mol %, and more preferably contains yttria in an amount of 3 to 10 mol % with respect to the total amount of yttria and zirconia.

In one embodiment, the biocompatible inorganic material is selected from the group consisting of titanium, silica, alumina, a cobalt-chromium alloy, stainless steel, and mullite. The cobalt-chromium alloy is a metal containing cobalt as a main component, 25% or more of chromium, 4% or more of molybdenum and other additives as main constituents, in which the total amount of cobalt, nickel, and chromium is 85% or more.

In one embodiment, the porous body for capturing cancer cells has communicating pores on a surface of the porous body for capturing cancer cells. The communicating pores are pores in which adjacent pores are connected to each other. Thus, the invasion of cancer cells to the inside of the pores of the porous body allows more cancer cells to adhere to the porous body. That is, this allows for capturing more cancer cells.

The surface property of the inner wall of the pore of the porous body of this embodiment is not limited, but one having a form with irregularities so as to increase the surface area of the porous body is desirable.

The porosity of the porous body of this embodiment is not limited, but is, for example, 40 to 95%, preferably 60 to 95%, and more preferably 80 to 95%. For the porosity of the porous body, a value measured by a method described in Examples is adopted.

The average pore diameter of the porous body of this embodiment is not limited as long as metastatic cancer cells can invade into the porous body. The average pore diameter of the porous body is, for example, 20 to 1000 μm, preferably 100 to 800 μm, and more preferably 300 to 500 μm. For the average pore diameter of the porous body, a value measured by a method described in Examples is adopted.

The ratio of open pores of the porous body of this embodiment is not limited, but is, for example, 20 to 90%., preferably 30 to 80%, and more preferably 35 to 50%. For the ratio of open pores of the porous body, a value calculated by a method described in Examples is adopted.

In the porous body of this embodiment, the type of cancer subjected to be captured is not limited. Examples thereof include nervous system cancers (for example, a brain tumor and neck cancer); digestive system cancers (for example, oral cancer, pharyngeal cancer, esophageal cancer, gastric cancer, hepatic cancer, gallbladder cancer, biliary tract cancer, spleen cancer, large intestine cancer, small intestine cancer, duodenal cancer, colon cancer, colon adenoma, rectal cancer, pancreatic cancer, and liver cancer); musculoskeletal cancers (for example, sarcoma, osteosarcoma, and myeloma): urinary system cancers (for example, bladder cancer and renal cancer); reproductive system cancers (for example, breast cancer, uterine cancer, ovarian cancer, testis cancer, and prostate cancer); respiratory system cancers (for example, lung cancer); hematopoietic cancers (for example, leukemia such as acute or chronic myeloid leukemia, acute promyelocytic leukemia, and acute or chronic lymphocytic leukemia, malignant lymphoma, (lymphosarcoma), hemangiosarcoma, multiple myeloma, myelodysplastic syndrome, primary myelofibrosis, hemangiopericytoma); thyroid cancer, parathyroid cancer, tongue cancer, malignant melanoma (melanoma), mastocytoma, cutaneous histiocytoma, lipoma, a hair follicle tumor, a skin papilloma tumor, sebaceous adenoma and basal cell cancer.

In one embodiment, the cancer cells are metastatic cancer cells. The metastatic cancer cells are not limited, but are, for example, metastatic cancer cells derived from the above-mentioned cancers, and preferably metastatic cancer cells derived from breast cancer, pancreatic cancer, lung cancer, liver cancer, and a brain tumor.

The shape and size of the porous body of this embodiment are not limited, and can be appropriately selected according to the application, intended use, site of use, or the like. Examples of the shape of the porous body include a spherical shape, a columnar shape, a plate-like shape, a rod-like shape, a cylindrical shape and a lattice shape (for example, a lattice structure).

(Production Method)

A method for producing the porous body for capturing cancer cells of this embodiment is not limited, but a known method for producing a porous ceramic can be appropriately referred to.

In one embodiment, the method for producing the porous body for capturing cancer cells of this embodiment includes the following steps:
(1) a step of preparing a dispersion by mixing a biocompatible inorganic material, a dispersant, a pore forming agent, and a dispersion medium:
(2) a step of obtaining a frozen body by filling the dispersion in a mold, followed by freezing;
(3) a step of obtaining a dry body by drying the frozen body;
(4) a step of degreasing the dry body; and
(5) a step of sintering the degreased dry body.

In the step (1), a dispersion is prepared by mixing a biocompatible inorganic material, a dispersant, a pore forming agent, and a dispersion medium.

For the biocompatible inorganic material, a powder of the above-mentioned biocompatible inorganic material can be used. The content of the biocompatible inorganic material is, for example, 20 to 50 mass %, and preferably 30 to 40 mass % with respect to the total mass of the dispersion.

The average particle diameter of the powder of the biocompatible inorganic material is, for example, 0.01 to 5 μm, and preferably 0.01 to 0.1 μm. The average particle diameter can be measured using a laser diffraction particle size analyzer.

The dispersant is used not only for contributing to dispersion of the biocompatible inorganic material, but also for forming communicating pores. As the dispersant, a water-soluble polymer can be used. Examples of such a water-soluble polymer include a polysaccharide and a derivative thereof, polyvinyl alcohol, a sodium salt of a polyacrylic acid or a polymethacrylic acid and polyethylene glycol. The water-soluble polymer is preferably a polysaccharide from the viewpoint of further exhibiting the effect of the present invention.

Examples of the polysaccharide include hydroxypropyl cellulose, carboxymethyl cellulose, agarose, methylcellulose, cellulose ether, hydroxyethyl cellulose, modified starch, dextran, alginic acid and sodium alginate. The polysaccharide is more preferably selected from hydroxypropyl cellulose, carboxymethyl cellulose, agarose, methylcellulose, cellulose ether and hydroxyethyl cellulose, and is particularly preferably hydroxypropyl cellulose.

The content of the dispersant is, for example, 0.5 to 5 mass %, and more preferably 1 to 3 mass % with respect to the total mass of the dispersion.

The pore forming agent has a function of forming pores. As the pore forming agent, for example, resin beads can be used. Examples of the resin beads include resin beads made of an acrylic resin or a urethane resin.

The shape of the pore forming agent is not limited, but is preferably a spherical shape.

The size of the pore forming agent is not limited, and can be appropriately selected so as to obtain desired pores.

The content of the pore forming agent is, for example, 30 to 60 mass %, and preferably 35 to 50 mass % with respect to the total mass of the dispersion.

The dispersion medium is preferably water. As the water, for example, tap water, distilled water, ion exchanged water, pure water, ultrapure water, or the like can be used. Among these, from the viewpoint of containing few impurities, the dispersion medium is preferably pure water or ultrapure water. Further, the dispersion medium may be one obtained by incorporating an inorganic salt in water.

The content of the dispersion medium is, for example, 15 to 40 mass %, and preferably 20 to 30 mass % with respect to the total mass of the dispersion.

A method for mixing the biocompatible inorganic material, the dispersant, the pore forming agent, and the dispersion medium is not limited, and a conventionally known findings can be appropriately referred to so that the biocompatible inorganic material and the pore forming agent are well dispersed in the dispersion. For example, a slurry in which the biocompatible inorganic material is uniformly dispersed is prepared by stirring and mixing the biocompatible inorganic material, the dispersant, and the dispersion medium. Heating may be performed as needed during the stirring and mixing. Thereafter, the pore forming agent is added to the prepared slurry, followed by mixing, and thus the dispersion can be prepared.

Adjusting the mixing proportion (mass proportion) of the biocompatible inorganic material, the dispersant, the pore forming agent and the dispersion medium allows for controlling the porosity, the ratio of open pores, the average pore diameter, or the like.

In the step (2), the dispersion prepared in the step (1) is filled in a mold, followed by freezing, and then a frozen body is obtained.

The shape, size, or the like of the mold is not limited, and can be appropriately selected according to the application of the porous body.

The material of the mold is not limited, and alumina, stainless steel, iron, copper, aluminum, a plastic, a paper, or the like can be used.

Freezing the dispersion filled in the mold causes ice crystals to grow and then causes a texture structure by the ice crystals to be formed. Accordingly, the frozen body containing a portion of the biocompatible inorganic material and the dispersant solution and a portion of the ice crystals can be obtained.

The freezing temperature can be appropriately selected according to the dispersion medium to be used. The freezing temperature is, for example, −10° C. or lower, and preferably −30° C. or lower.

In the step (3), the frozen body obtained in the step (2) is dried to obtain a dry body. Drying the frozen body (for example, freeze drying or vacuum drying) causes the ice crystals to be sublimated and disappear to form pores in place of ice crystals.

In the step (4), the dry body obtained in the step (3) is degreased. In this step, organic components such as the dispersion medium and the pore forming agent are removed from the dry body. Specifically, the organic components are decomposed and removed under the condition of a predetermined temperature according to the type of the biocompatible inorganic material to be used. The degreasing temperature is, for example, 300 to 900° C. The degreasing time can be appropriately adjusted so that the organic components can be removed by decomposition.

In the step (5), the degreased dry body is sintered. The sintering temperature, the sintering time, the sintering atmosphere, or the like can be appropriately adjusted according to the biocompatible inorganic material to be used, the porosity, or the like. The sintering temperature is, for example, 1000 to 1700° C. when yttria-stabilized zirconia is used as the biocompatible inorganic material, and 1000 to 1700° C. when alumina is used as the biocompatible inorganic material.

In this manner, the porous body according to the present invention can be produced.

(Application)

The porous body for capturing cancer cells of this embodiment can be used for application related to cancer such as a treatment, a treatment assistance, a test, or a diagnosis. In particular, the porous body for capturing cancer cells of this embodiment is suitable for application of a treatment or a treatment assistance. Specifically, the porous body for capturing cancer cells of this embodiment can be used for capturing metastatic cancer cells by being implanted in the body of a subject.

The present inventors found that cancer cells adhere to the porous body of this embodiment (the below-mentioned Test Examples 1 and 2). Then, it was confirmed that an adverse event attributed to the porous body does not occur, based on an experiment in which the porous body of this embodiment is implanted in a mouse (the below-mentioned Test Example 3). Further, it was confirmed that a life extension effect is obtained after a primary lesion is resected in a cancer-bearing mouse implanted with the porous body of this embodiment (the below-mentioned Test Example 4).

One embodiment of the present invention relates to a porous body for capturing cancer cells for being implanted in a subject, which contains a biocompatible inorganic material.

Further, one embodiment of the present invention relates to use of a porous body for capturing cancer cells, preferably for capturing cancer cells by implanting the porous body in a subject, wherein the porous body contains a biocompatible inorganic material.

In the present description, the “subject” is, for example, a mammal, a bird, or the like, and is preferably a mammal and a bird that has cancer or is suspected to have cancer. Here, the mammal includes both primates such as a human, a monkey, a gorilla, a chimpanzee and an orangutan, and non-human mammals such as a mouse, a rat, a hamster, a guinea pig, a rabbit, a dog, a cat, a pig, cattle, a horse, sheep, a camel and a goat. Examples of the bird include a chicken, a quail and a pigeon. Among these, a human, a hamster, a guinea pig, a rabbit, a dog, a cat, and a pig are preferred, and a human or a pet animal such as a dog, a cat, or a rabbit is more preferred.

A site where the porous body for capturing cancer cells of this embodiment is implanted (embedded) is not limited, but is preferably a subcutaneous, intramuscular, intraperitoneal, or tumor resected site. Further, the porous body for capturing cancer cells can be implanted at one or more sites of a subject.

<Implant for Capturing Cancer Cells>

As described above, the porous body for capturing cancer cells of this embodiment can be used by being implanted in a subject. Therefore, one embodiment of the present invention relates to an implant for capturing cancer cells containing the above-mentioned porous body for capturing cancer cells.

The implant for capturing cancer cells is preferably used for capturing metastatic cancer cells in a subject by being implanted in the subject.

In the present description, the “implant” means an instrument to be implanted in a subject by a surgical method or a low invasive method, more specifically, an instrument to be completely or partially implanted in the body of a subject.

In one embodiment, the implant for capturing cancer cells includes the above-mentioned porous body for capturing cancer cells.

A site where the implant for capturing cancer cells of this embodiment is implanted is not limited, but is preferably a subcutaneous, intramuscular, intraperitoneal, or tumor resected site. Further, the implant for capturing cancer cells can be implanted at one or more sites of a subject.

The implant for capturing cancer cells may further contain a drug in addition to the porous body for capturing cancer cells. In the present description, the “drug” means a compound that can be used for therapeutic purpose.

Examples of the drug include a protein drug, an alkylating agent, an antibiotic, an antimetabolite, a hormone preparation, a platinum preparation, a topoisomerase inhibitor, a microtubule inhibitor and an angiogenic inhibitor.

Examples of the protein drug include a peptide, an enzyme, a structural protein, a receptor, a cytokine, a chemokine, a hematopoietic factor and a growth factor. Specific examples thereof include IL-1, IL-4, IL-8, IL-10, IL-13, IL-17, CCL2, CCL5, CCL9, CCL18, CCL19, CCL20, CCL21, CCL25, CCL27, CCR4, CCR5, CCR7/CCL21, CCR9, CCR10, CCL18, CCL2/MCP-1, MIP-1α/CCL3, CXCL1, CXCL2, CXCL3, CXCL4, CXCL5, CXCL8, CXCL12/SDF-1α, CXCR2, CXCR3, CXCR4, CXCR7, erythropoietin (EPO), CCL5/RANTES, a hepatocyte growth factor activator (HGFA), insulin-like growth factor-1 (IGF-1), cyclooxygenase-2 (COX-2), CXCL14, prostaglandin E2, a platelet-derived growth factor and a vascular endothelial growth factor (VEGF).

Examples of the alkylating agent include a mustard, a nitrosourea, a triazene and ethylenimine. More specifically, cyclophosphamide, ifosfamide, busulfan, melphalan, nitrogen mustard, chlorambucil, glufosfamide, mafosfamide, estramustine, nimustine, ranimustine, carmustine, lomustine, semustine, streptozocin, procarbazine, dacarbazine, temozolomide, thiotepa, hexamethylmelamine, trabectedin and apaziquone, altretamine, bendamustine, mitolactol.

Examples of the antibiotic include an anthracycline-based antibiotic (for example, doxorubicin (adriamycin), daunorubicin, pirarubicin, epirubicin, idarubicin, aclarubicin, amurubicin, zorubicine, valrubicin, liposomal doxorubicin, pixantrone, mitoxantrone, and the like), mitomycin C, bleomycin, peplomycin, actinomycin D and zinostatin stimalamer.

Examples of the antimetabolite include a pyrimidine analog, a purine analog, a folic acid analog, a ribonucleotide reductase inhibitor and a DNA polymerase inhibitor. More specific examples of the antimetabolite include fluorouracil (5-FU), tegafir, a tegafur/uracil combined drug, a tegafur/gimeracil/oteracil potassium combined drug, doxifluridine, capecitabine, carmofur, cytarabine, cytarabine ocfosfate, enocitabine, gemcitabine, azacitidine, decitabine, floxuridine, ethynylcytidine, 6-mercaptopurine, fludarabine, pentostatin, nelarabine, 6-thioguanine, cladribine, clofarabine, methotrexate, pemetrexed, raltitrexed, nolatrexed, pralatrexate, trimetrexate, edatrexate and hydroxycarbamide.

Examples of the hormone preparation include an antiestrogen, an aromatase inhibitor, a progesterone derivative, an antiandrogen, a corticosteroid and an LHRH (luteinizing hormone-releasing hormone) agonist. More specific examples of the hormone preparation include tamoxifen, toremifene, raloxifene, fulvestrant, anastrozole, exemestane, retrozole, aminoglutethimide, formestane, vorozole, methyltestosterone, medroxyprogesterone, megestrol, gestonorone, mepitiostane, flutamide, nilutamide, bicalutamide, finasteride, chlormadinone, estramustin, diethylstilbestrol, ethinylestradiol, fosfestrol, polyestradiol phosphate, prednisolone, dexamethasone, mitotan, goserelin, leuprorelin, buserelin and triptorelin.

Examples of the platinum preparation include cisplatin, carboplatin, nedaplatin, oxaliplatin (Ox), satraplatin, miriplatin, lobaplatin, spiroplatin, tetraplatin, ormaplatin and iproplatin.

Examples of the topoisomerase inhibitor include a topoisomerase I inhibitor and a topoisomerase II inhibitor. More specific examples of the topoisomerase inhibitor include topotecan, irinotecan, exatecan, nogitecan, the above-mentioned anthracycline-based antibiotic, etoposide, teniposide and sobuzoxane.

Examples of the microtubule inhibitor include a vinca alkaloid and a taxane compound. More specific examples of the microtubule inhibitor include vincristine, vinblastine, vindesine, vinorelbine, vinflunine, monomethyl auristatin E, epothilone B, eribulin, paclitaxel (taxol), docetaxel (DTX) and cabazitaxel.

Examples of the angiogenic inhibitor include a VEGF (vascular endothelial growth factor) inhibitor such as an anti-VEGF antibody, an angiogenesis signaling inhibitor such as a tyrosine kinase inhibitor and an MMP (matrix metalloproteinase) inhibitor. More specific examples of the angiogenic inhibitor include bevacizumab, aflibercept, MV833, cetuximab, pegaptanib, pazopanib, CBO-P11, sunitinib, sorafenib, ranibizumab, vatalanib, axitinib, Zactima, NX1838, Angiozyme, semaxanib, lestaurtinib, TSU-68, ZD4190, temsirolimus, angiostatin, endostatin, TNP-470, CP-547632, CPE-7055, KRN633, AEE788, IMC-1211B, PTC-299, E7820, CC, Marimastat, Neovastat, Prinomastat, Metastat, BMS-275291, MM1270, S-3304, vitaxin, carboxyamidotriazole orotate, thalidomide, genistein, interferon-α and interleukin-12.

<Method for Capturing Metastatic Cancer Cells>

One embodiment of the present invention relates to a method for capturing metastatic cancer cells including implanting the above-mentioned implant for capturing cancer cells in the body of a subject.

The respective comfigurations related to this embodiment are the same as those for the porous body for capturing cancer cells and the implant for capturing cancer cells, and therefore, the description thereof will be omitted.

### EXAMPLES

Hereinafter, the embodiments of the present invention will be described in more detail with reference to Examples, but the technical scope of the present invention is not limited to the following Examples.

### Production of Porous Body for Capturing Cancer Cells: Example

To a mixed solution of 5 g of ultrapure water and 0.5 g of hydroxypropyl cellulose, 12.1 g of an yttria-stabilized zirconia powder (TZ3Y, Tosoh Corporation) was added, followed by heating and stirring using a hot plate stirrer (60 to 70° C.). After the stirring, 15 g of resin beads (S-40, Sekisui Kasei Co., Ltd.) were added to the solution and then the resultant solution was mixed. The resulting mixture was filled in an alumina ring (tube) (diameter: 9.0 mm, thickness: 2.7 mm), and frozen at −30° C., and then, dried for 24 hours using a freeze dryer. After the dried one was degreased at 800° C. for 2 hours (temperature increasing rate: 1° C./min) using an electric furnace in an air atmosphere, the resultant one was sintered at 1400° C. for 6 hours (temperature increasing rate: 5° C./min) using the electric furnace, and thus a porous body for capturing cancer cells (porous scaffold, diameter: 6.7 mm, thickness: 2.1 mm) was produced.

(Measurement of Porosity)

The volume (calculated by actual measurement of the diameter and the thickness) and weight of the produced porous body for capturing cancer cells were determined, and a porosity was calculated from a ratio of the weight of the porous body for capturing cancer cells to the weight of a dense body having the same volume calculated from the specific gravity of yttria-stabilized zirconia.

The porosity of the produced porous body for capturing cancer cells was 85%.

(Calculation of Average Pore Diameter)

The diameters of the pores observed from an electron microscopy (scanning electron microscopy: SEM) image were measured, and an average pore diameter was calculated from the measured values.

The average pore diameter of the produced porous body for capturing cancer cells was 400 μm.

(Calculation of Ratio of Open Pores)

The area of the opening portion was measured from an electron microscopy (scanning electron microscopy: SEM) image, and the area ratio of the opening portion per unit area was determined as a ratio of open pores.

The ratio of open pores of the produced porous body for capturing cancer cells was 50%.

### Production of Smooth and Dense Scaffold: Comparative Example

After 0.8 g of an yttria-stabilized zirconia powder (TZ3Y, Tosoh Corporation) was packed in a press jig having a diameter of 17.5 mm, the packed one was uniaxially press-molded (200 MPa, 1 min), and then the press-molded one was subjected to cold isostatic pressing (200 MPa, 1 min). The resultant one was sintered at 1400° C. for 1 hour (temperature increasing rate: 5° C./min) using an electric furnace in an air atmosphere, and thus a smooth and dense scaffold (diameter: 13.5 mm, thickness: 1.2±0.2 mm) was produced.

<Preparation of Cell Suspension>

4T1 cells derived from mouse breast adenocarcinoma were cultured in a culture medium (RPMI 1640 medium containing 10% FBS), and then a cell suspension was prepared.

### Test Examples

1. Evaluation of Cell Adhesion to Porous Body for Capturing Cancer Cells In Vitro

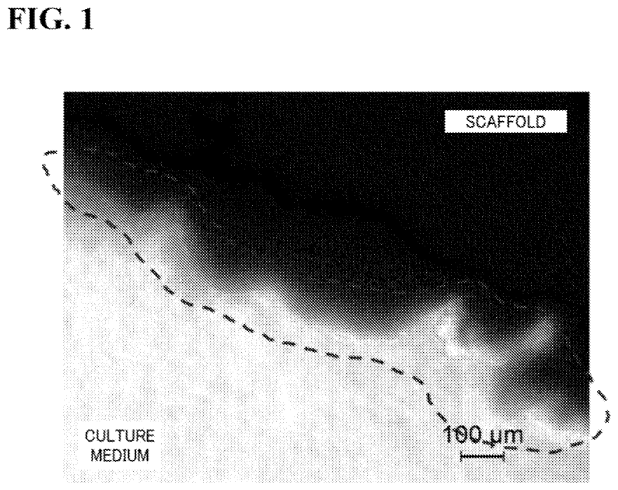

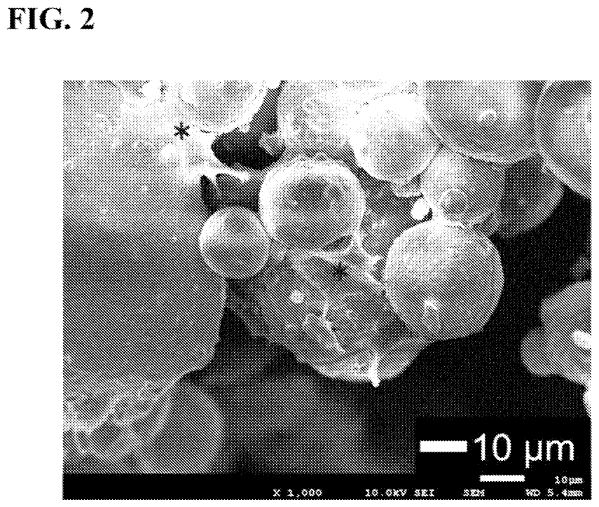

2 mL of the cell suspension at 1.0×10⁶ cells/mL was inoculated in a culture dish having a diameter of 35 mm. The sterilized porous body for capturing cancer cells was immersed in the cell suspension. Thereafter, the cells were incubated for 2 hours at 37° C. in a 5% (v/v) CO₂ environment, and then, the porous body for capturing cancer cells was transferred to another culture dish, and 2 mL of a culture medium (RPMI 1640 medium containing 10% FBS) was added thereto. Then, the cells were cultured for 1 week at 37° C. in a 5% (v/v) CO₂ environment. After the culture, the state of cell adhesion was observed using an optical microscope (magnification: 100-fold). Further, the porous body for capturing cancer cells after the cell culture was fixed and freeze-dried, and then, the fixed and freeze-dried porous body was observed using a scanning electron microscope (magnification: 1000-fold). The results were shown in FIGS. 1 and 2.

As shown in FIG. 1, it was found that the cancer cells adhered to the side face of the porous body for capturing cancer cells in a large amount. Further, as shown in FIG. 2, it was found that the cancer cells adhered to the cross section (inside) of the porous body for capturing cancer cells by extending pseudopodia of the cancer cells.

2. Evaluation of Necessity of Porous Face for Cell Adhesion

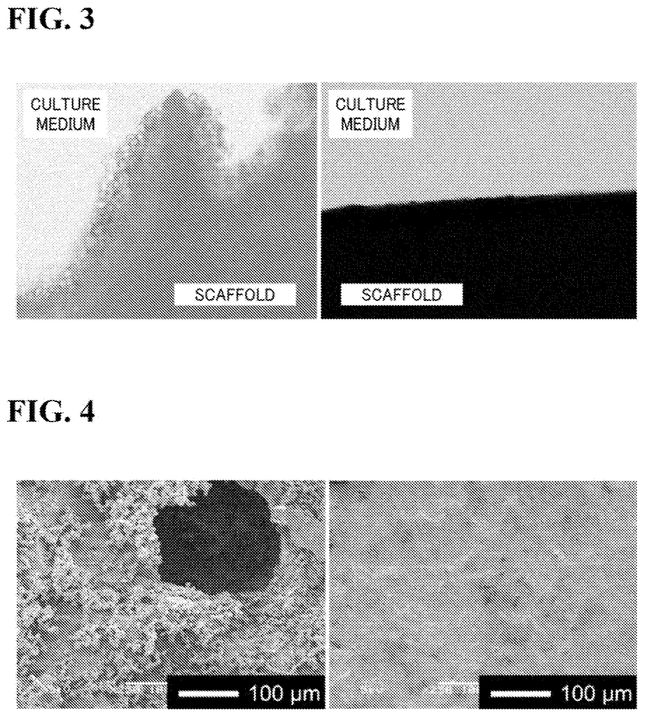

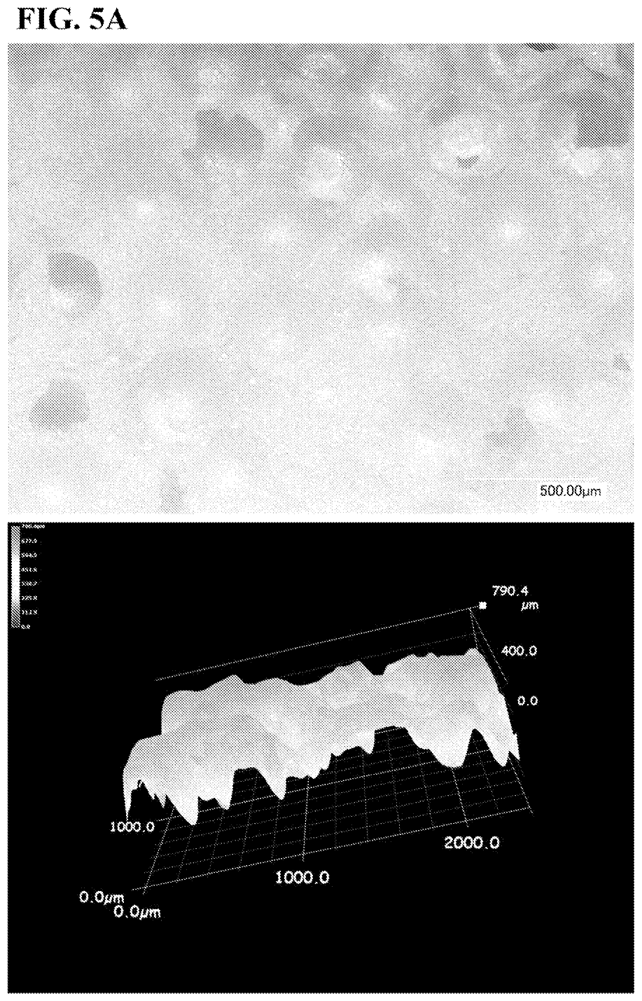

500 μL of the cell suspension at 1.0×10⁶ cells/mL was added dropwise on the sterilized porous body for capturing cancer cells or the sterilized smooth and dense scaffold. Thereafter, the cells were incubated for 2 hours at 37° C. in a 5% (v/v) CO₂ environment, and then, the porous body for capturing cancer cells or the smooth and dense scaffold was transferred to another culture dish, and 2 mL of a culture medium (RPMI 1640 medium containing 10% FBS) was added thereto. Then, the cells were cultured for 1 week at 37° C. in a 5% (v/v) CO₂ environment. After the culture, the state of cell adhesion was observed using an optical microscope (magnification: 100-fold). Further, the porous body for capturing cancer cells or the smooth and dense scaffold after the cell culture was fixed and freeze-dried, and then the fixed and freeze-dried one was observed using a scanning electron microscope (magnification: 1000-fold). The results were shown in FIGS. 3 and 4.

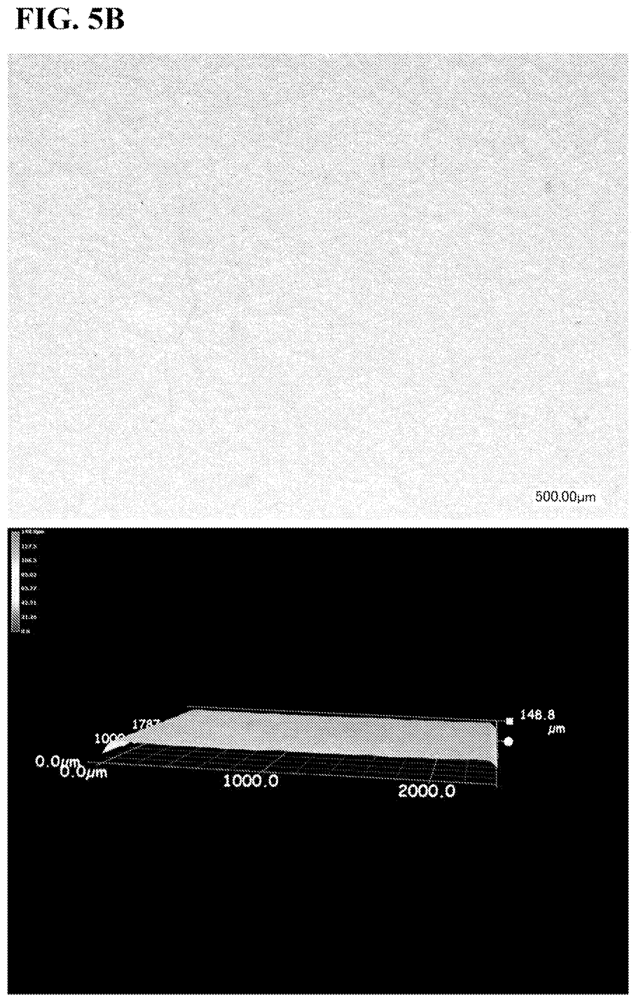

In addition, the surfaces of the porous body for capturing cancer cells and the smooth and dense scaffold before the test were observed using an optical microscope (magnification: 100-fold), and further, the surface roughness was measured using a stereoscopic microscope (manufactured by Keyence Corporation). The results were shown in FIGS. 5A and 5B.

As shown in FIG. 3, it was found that the cancer cells adhered to the side face of the porous body for capturing cancer cells in a large amount. On the other hand, it was found that the cancer cells hardly adhered to the side face of the smooth and dense scaffold.

Further, as shown in FIG. 4, it was found that the cancer cells adhered to the cross section (inside) of the porous body for capturing cancer cells in a large amount. On the other hand, it was found that the cancer cells hardly adhered to the side face of the smooth and dense scaffold.

As shown in FIGS. 5A and SB, it was found that the porous body for capturing cancer cells was porous, and therefore the surface is a rough surface, on the other hand, the smooth and dense scaffold has a dense and smooth face. Therefore, from FIGS. 3 to 5B, it was found that being porous and having a rough face was effective in cell adhesion.

3. Evaluation of Safety of Implantation in Mouse In Vivo

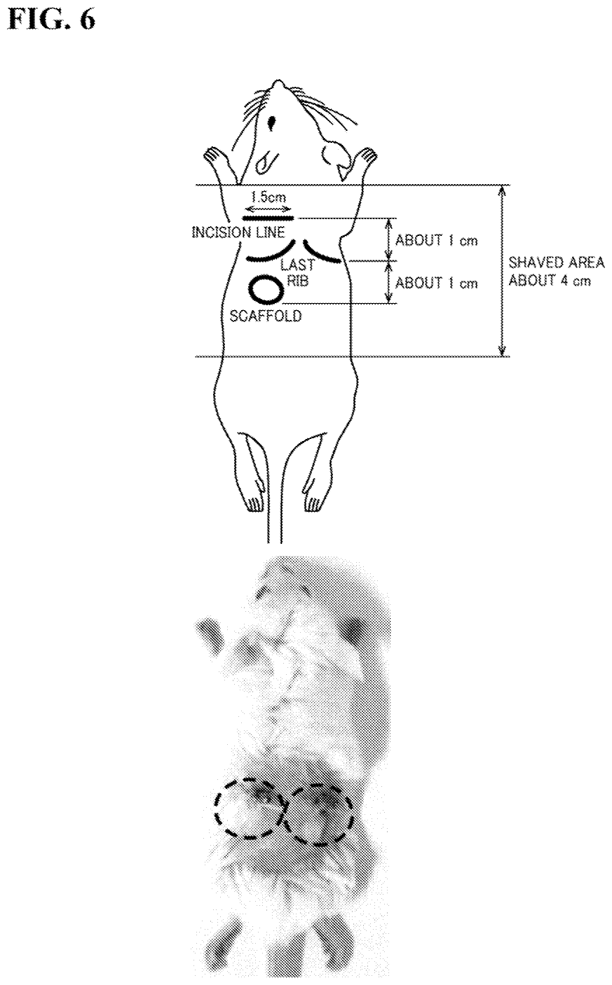

The back of a mouse at 8 weeks of age (BALB/c, purchased from Japan SLC Co., Ltd.) under general anesthesia was shaved according to FIG. 6. A portion 1 cm from the edge of the last rib on the head side was incised 1.5 cm along the short axis using a surgical knife. The tissue under the skin was detached from the incision line to the tail side, and the scaffold was inserted from the incised portion and placed therein, and thereafter, the incised portion was sutured. Also, for the other side, the same operation was performed. The implanted state was observed for up to a maximum of 2 months, and it was confirmed by visual observation that there was no abnormality in the physical condition of the mouse and there was no abnormality in the implantation site.

Note that the animal experiment using the mouse was performed based on the provisions of Articles 8, 9, and 11 of Osaka Prefecture University Animal Experiment Regulations (Approval number: Animal Experiment No. 19-165).

4. Evaluation of Life Extension Effect on Cancer-Bearing Mouse Implanted with Porous Body for Capturing Cancer Cells

The porous body for capturing cancer cells was implanted in a mouse in the same manner as in the above Item 3. Cancer cells were transplanted according to the following procedure 1 month after the implantation.
(1) An abdominal portion of the mouse under general anesthesia was shaved;
(2) a portion moved about 5 cm from the base of a right hind limb to the body axis side was incised 1 cm along the long axis;
(3) the fourth mammary adipose tissue was exposed from the incised portion;
(4) a suspension of 4T1 cells derived from mouse breast adenocarcinoma filled in a tuberculin syringe was injected into the mammary gland fat pad; and
(5) the mammary gland fat was returned under the skin and the incised portion was sutured.

10 days after the transplantation of the cancer cells, the mammary gland portion of the mouse under general anesthesia was incised in the same manner as the procedure (2), and a primary lesion was resected, and thereafter, the incised portion was sutured.

The mouse was bred while observing the physical condition, and the survival period was compared with the control (no scaffold implantation) group (each group n=20, Date of resection of primary lesion: 0 day). However, a mouse in which regrowth was observed in the primary lesion was excluded from the analysis.

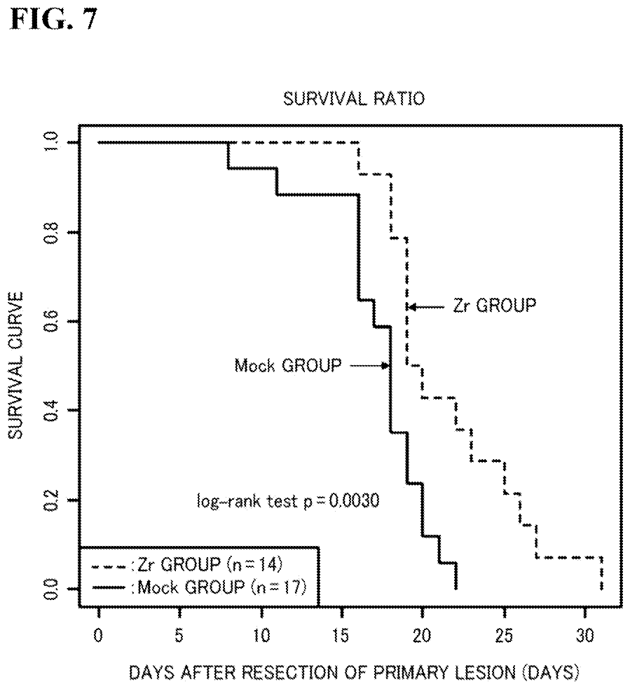

The results were shown in FIG. 7. In FIG. 7, Zr group is a mouse group in which the porous body for capturing cancer cells was implanted, and Mock group is the control group.

As shown in FIG. 7, it was found that the life extension effect in the cancer-bearing mouse was obtained by implanting the porous body for capturing cancer cells of Example.

The present application is based on Japanese Patent Application No. 2020-198470 filed on Nov. 30, 2020, the contents of which are incorporated herein by way of reference.

### INDUSTRIAL APPLICABILITY

A porous body for capturing cancer cells according to an embodiment of the present invention has biocompatibility and also has stability in a living body.

1. A porous body for capturing cancer cells, comprising a biocompatible inorganic material.

2. The porous body for capturing cancer cells according to claim 1, wherein the biocompatible inorganic material is yttria-stabilized zirconia.

3. The porous body for capturing cancer cells according to claim 1, wherein the biocompatible inorganic material is selected from the group consisting of titanium, silica, alumina, a cobalt-chromium alloy, stainless steel, and mullite.

4. The porous body for capturing cancer cells according to claim 1, comprising communicating pores.

5. The porous body for capturing cancer cells according to claim 1 wherein a porosity of the porous body is 40 to 95%.

6. The porous body for capturing cancer cells according to claim 1 wherein an average pore diameter of the porous body is 20 to 1000 μm.

7. The porous body for capturing cancer cells according to claim 1, wherein a ratio of open pores of the porous body is 20 to 90°.

8. The porous body for capturing cancer cells according to claim 1 wherein the cancer cells are metastatic cancer cells.

9. An implant for capturing cancer cells, comprising the porous body for capturing cancer cells according to claim 1.

10. The implant for capturing cancer cells according to claim 9, wherein the implant is used for being implanted to a subject and for capturing metastatic cancer cells in the subject.

11. A method for capturing metastatic cancer cells, comprising implanting the implant for capturing cancer cells according to claim 9 into a body of a subject.

12. An use of a porous body for capturing cancer cells, wherein the porous body according to claim 1 is used.
